(19) United States
(12) Patent Application Publication
(10) Pub. No.: US 2017/0087010 A1
(43) Pub. Date: Mar. 30, 2017

(54) Temperature controlled hypoallergenic garments or pads

(71) Applicant: Kanita Anjanette Howard, Washington, DC (US)

(72) Inventor: Kanita Anjanette Howard, Washington, DC (US)

(21) Appl. No.: 14/756,689

(22) Filed: Sep. 30, 2015

(51) Int. Cl. A61F 7/02; A61L 31/16; A61L 15/16; A61L 31/04; A61L 31/14; A61L 24/00

(52) U.S. Cl. CPC A61F 7/0241; A61L 31/14; A61L 24/00; A61L 15/16; A61L 31/042; A61L 31/16; A61F 2007/005

(57) ABSTRACT

A hypoallergenic garment is provided that offers relief by achieving a desirable cooling or heating effect in an individual experiencing pain or discomfort. It can be used for treatment of conditions such as itching, burning, yeast or bacterial infection, urethral pain, inflammation, urinary tract infections, dermatitis, vaginal varicosity, menopausal perspiration, post-surgery swelling, edema due to a sports injury, swelling due to trauma, damage to the perineum, menopausal pain and combinations thereof.

### FIELD OF THE INVENTION

This invention relates generally to the field of temperature control garments and pads for a human or animal user.

### BACKGROUND OF THE INVENTION

The following discussion of the background of the invention is merely provided to aid the reader in understanding the invention and is not admitted to describe or constitute prior art to the present invention.

Throughout history, people have used various methods in attempts to keep themselves comfortable in different environments, especially when engaged in strenuous activities.

This issue affects women more severely as removing clothing is often inappropriate or undesirable while changing into clothing is often inconvenient or inappropriate. Similarly, wetting the body and/or clothing can be uncomfortable, unaesthetic, or even embarrassing. Often, people find it impossible to feel comfortable or cool when wearing heavy or protective clothing or equipment. The ambient temperatures of some work environments are beyond their control, and can be quite uncomfortable. By way of example, a surgeon working in an operating room may have little control over room temperature. To make matters worse, while a woman may have little control over the ambient temperature, she may also have little control over her own wardrobe. Often, for example, a person is required to wear a particular uniform. There is thus a need for improved garments capable of selectively cooling or heating the wearer.

Common problem experienced by individuals is pain or discomfort in the groin and/or adjacent regions. Specifically in women, pain or discomfort can be in the vulva, perineum and surrounding regions due to, but not limited to, itching and burning as a result of infection (e.g., bacterial or yeast infection), urethral pain or inflammation as a result of infection (e.g., urinary tract infections), vaginal varicosity (e.g., during pregnancy), damage to the perineum following childbirth, and chronic inflammation (e.g., dermatitis). However, this invention is equally applicable to a man or child.

While various ways of cooling or warming the groin region have been proposed, none have gained widespread acceptance in the market place. Perhaps this is due to inconvenience in the mode of application and/or poor design, causing additional discomfort. Cooling or waring garments, cooling pads or cooling devices for the perineum that have been proposed have various design aspects that limit their usefulness and effectiveness. Hence, there remains a need to provide such comfort.

The instant invention, while overcoming the limitations of these proposed devices, pads and garments, comprises garments (including undergarments) and pads for a groin area. More specifically, the garment has pockets or other means for accommodating the insertion of cooling pads or packs in selected areas for the localized application of cooling. In the case of women, the inventor's undergarment can be used to treat conditions selected from the group consisting of itching, burning, yeast or bacterial infection, urethral pain, inflammation, urinary tract infections, dermatitis, vaginal varicosity, menopausal perspiration, post-surgery swelling, swelling due to trauma, damage to the perineum and combinations thereof.

### DETAILED DESCRIPTION OF THE INVENTION

In this specification, although the preferred embodiments have been described in detail, it should be understood that various changes, substitutions and alterations might be made therein without departing from the spirit and scope of the invention. Therefore, the specification is to be regarded in an illustrative rather than a restrictive sense.

All references, including publications, patent applications, and patents, cited herein are incorporated by reference in full to the same extent as if each individual publication was specifically and individually indicated to be incorporated by reference. They are indicative of the levels of those of ordinary skill in the art to which the invention pertains and may be employed in the practice of the invention.

The invention illustratively described herein suitably may be practiced in the absence of any element or elements, limitation or limitations, which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed.

As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” In other words, the use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Relational terms such as first and second, top and bottom, and the like may be used solely to distinguish one entity or action from another entity or action without necessarily requiring or implying any actual such relationship or order between such entities or actions. Recitation of ranges of values herein are merely intended to serve as a shorthand method of referring individually to each separate value falling within the range, unless otherwise indicated herein, and each separate value is incorporated into the specification as if it were individually recited herein. Cool temperatures within a refrigerator are typically between 33-45° F. (0.5 to 10° C.). Cold temperatures within a freezer are typically between 0 to 32° F. (−20 to 0° C.). Room temperature is generally considered to be around 25° C. All methods described herein can be performed in a suitable order unless otherwise indicated herein or otherwise clearly contradicted by context.

The present invention provides garments and pads, more specifically, undergarment that are suitable for wearing in lieu of briefs, panties or thong underwear, to achieve a desirable cooling effect. Terms such as pad and pack are interchangeably used in this specification while the cooling gel is material contained therein. Various advantages and features of the present invention will become more fully apparent from the following description and appended claims, or may be learned by the practice of the invention as set forth hereinafter.

In one embodiment of the instant invention, the garment is an undergarment that is pre-adherent to the wearer, e.g., having pre-applied adhesive material on the body side, suitable for contact with skin and sufficiently adherent to removably hold the undergarment in place during use. That is, the undergarment, packaged for storage and/or shipment, sale, etc., carries pre-applied adhesive on the body side (the side contacting the wearer's vulva and/or skin surrounding the vulva). The adhesive layer may comprise a variety of different adhesive materials known in the art. The pre-adherent adhesive may be pre-applied to some or all of the body side of the undergarment. With such pre-applied adhesive, the undergarment in certain exemplary embodiments can be applied onto the body by the wearer or an assistant after removal of any cover, e.g., a disposable sheet or envelope, simply by positioning the undergarment and applying gentle pressure. In certain exemplary embodiments, the pre-applied adhesive is a pressure sensitive or contact adhesive, such that the undergarment can be applied by positioning it and applying gentle pressure. In certain exemplary embodiments, the adhesive is moisture and/or heat activated, such that the undergarment can be applied by holding it in position for a short time, in some embodiments following pre-wetting of the adhesive with water. Suitable adhesives are commercially available and their use in the minimalist undergarments disclosed here would be readily apparent to those of ordinary skill in the undergarment art given the benefit of this disclosure. The adhesive may be uniform or non-uniform in thickness. In certain exemplary embodiments of the undergarments disclosed here, it is suitable for contact with human skin, optionally for prolonged contact with human skin, e.g., for contact for more than an hour. Certain exemplary embodiments are suitable for contact up to 2-6 hours.

In another aspect of the invention, said garment comprises a removable protective cover over at least a portion of the adhesive. In accordance with a first aspect of the invention, garments are disclosed for wearing instead of or in addition to more traditional garments well known in the art.

As discussed earlier, the key feature of the present invention comprises different styles of garments with compartment(s) for placement of a gel that is not only thin to be discreet but is also extremely comfortable to the wearer's shape. The garment can be placed in the freezer or microwave and once it is ready for use, it can be inserted into the compartment in the lining of the garment. The garment may be made of a material that absorbs sweat and other body fluids away from the skin. The material would absorb the perspiration or sweat, lock it in and feel dry to the touch.

In yet another embodiment of the present invention, various additional inserts for the gels could be present in the garments. For instant, there could additionally be a cooling area located in the front of the garment. In other words, these so-called cooling or warming pads mechanisms can be inserted in the garment. The pads also come with a discreet storage container that can be placed in the freezer or microwave prior to use.

One feature of the garment can involve regulating the amount of cold or heat applied to the wearer; in one embodiment, the various compartments or pockets of the garment include a thermal adjustment layer. This thermal adjustment layer, which can be another pocket itself, a foldable layer of material, or other suitable structure, may be placed in varying locations relative to the pad so as to help with temperature regulation. For instance, in one mode of the invention, the thermal adjustment layer may be placed beneath the pad to reduce the amount of cooling or heat felt by the wearer. When the thermal adjustment layer is moved atop the pad, the wearer experiences increased thermal sensation, as fewer layers of material are present between the pad and the wearer's body.

Certain embodiments of said pad provide relief and comfort for an individual experiencing pain or discomfort of the groin and/or adjacent regions. According to one embodiment, a self-contained pad that requires no special manipulation or loading prior to use is provided. The pad of this embodiment includes a gel pad that can be placed in a cooling or warming environment prior to use, and then removed from the cooling or warming environment and immediately placed over the groin and/or adjacent region to provide relief and comfort without manipulation.

The gel is advantageously smooth, flexible and/or moldable to the wearer's shape in order to maximize comfort and minimize/eliminate lumps or protruding regions. Obviously, such a gel that is smooth, flexible, and/or moldable and is far more comfortable to wear and can more evenly distribute the cooling or warming action to the groin and/or adjacent region. In this regard, the instant invention contrasts to products on the market that utilize ice chunks for cooling. Furthermore, this contrasts to marketed chemical cooling media (e.g., those cooled via a chemical reactions between water and a salts like thiosulfates having a positive enthalpy of dissolution), in that a cooling gel that is cooled in a cooling environment prior to use can provide greater cooling and/or cooling for longer periods of time.

According to another embodiment, said garment is provided that includes an absorbent layer designed to be placed against a non-groin area, a temperature controlling layer adjacent to the absorbent layer, and an adhesive layer (discussed above) adjacent to the temperature controlling layer designed to adhere the cooling pad to an garment of the user. Such an arrangement keeps the gel pad in the desired position while eliminating the need for cumbersome attachment mechanisms, thereby enhancing comfort and promoting compliance. The adhesive layer may include a removable backing layer that protects the adhesive prior to use and which can be conveniently removed by the female user prior to use. Eliminating cumbersome attachment mechanisms, belts or harnesses, also results in a less visually noticeable garment beneath ordinary clothing, providing enhanced assurance and emotional well-being so that the product can be worn in public with minimal embarrassment compared to products that produce unsightly bulges and lines.

According to a specific embodiment, said pad is elongated to provide cooling to the entire groin and/or adjacent region. The pad can also extend against the woman's perineum to provide cooling and relieve to the perineum following childbirth or extend beyond the perineum and over the anal region to provide cooling and relief to that region if desired. However, it is to be understood that the shape of the pad may be performed or cut into any desired size or shape for placement to any appropriate area of the individual's body.

An advantage of the instant pad is that it is relatively flat just prior to use, and this permits the application (or insertion into the pocket in the garment) of several pads to be stacked on top of each other simultaneously prior to use. It also, makes it possible to place the container containing these gel pads directly into a refrigerator or freezer after purchase and an individual cooling pad withdrawn from the container as needed prior to use. This maximizes convenience to the user. The pads are advantageously disposable in order to maximize cleanliness to the user and eliminate the need to clean and reuse the product. As a specific example, pre-curved orientations that approximate the curvature and shape of the region of a woman's body to which the product is designed to cover. For example, an elongated product can have an approximate U-shaped configuration in order to conform to the front and back of a woman's crotch region and thereby minimize the amount of flexing that is required to conform the cooling pad to the woman's body prior to use.

Various compositions of the cooling gel that comprises the cooling pad are within the scope of this invention. An example of a cooling gel composition includes water, salt and a gelling agent (hydroxy-ethyl cellulose). The gel may also include various colorants, preservatives and/or anti-freeze (ethylene glycol, propylene glycol, or isopropyl alcohol) to soften the cooled gel and render it more flexible or moldable after being cooled in a freezer. The amount of anti-freeze can be varied to provide the desired level of flexibility and/or moldability when cooled to freezing or below freezing temperatures. In certain instances, it may desirable to allow congealing of the water as allowing water to undergo a phase change may increase the overall ability of the gel to provide a desired cooling effect.

According to yet another embodiment of the present invention, the corners of the cooling pad may be rounded in order to prevent them from poking or otherwise irritating the user's body during placement and use. This may be particularly useful in the case where the cooling layer becomes rigid when frozen. Obviously, other configurations are within the scope of this invention; the idea is to maximize comfort to the user. In the case where the pad is desired to wrap around a user's groin area, it may be desirable for the cooling pad to have a curved (e.g., V-shaped) configuration prior to use, thereby reducing or eliminating the amount of bending that must be performed during placement of the relatively rigid device. This, in turn, maximizes compliance by the user. Furthermore, the pads can have a variety of different sizes and/or thicknesses to provide a desired level of comfort, cooling ability and/or coverage. According to one preferred embodiment, the pad may have a length of about 6 inches and a width of about 2 inches. According to another preferred embodiment, the pad may have a length of about 9 inches and a width of about 3 inches.

As indicated earlier, the garments containing the pads disclosed herein can be used to alleviate pain and discomfort caused by a wide variety of ailments, such as pain and discomfort of the vulva, perineum, and surrounding regions. Such discomfort can have a variety of causes, including but not limited to, itching and burning as a result of infection (e.g., yeast or bacterial infection), urethral pain or inflammation as a result of infection (e.g., urinary tract infections), chronic inflammation (e.g., dermatitis, etc.), menopausal pain, pain due to sports, vaginal varicosity involving formation of varicose veins in the vulva or vagina during pregnancy due to increased pressure by the fetus that prevents adequate blood drainage, damage to the perineum following child birth, etc.

In another embodiment, the present invention is directed to a kit of one or more reusable gel pads (e.g., 5-20) which allows insertion of the gel pad into the pocket in the garment prior to use. A closure means can be used to retain the pad within the compartment of the garment during use. In the case when the user decides to use a fresh pad, the pad is placed into the freezer or microwave, depending on the specific application desired, and the cold or warm gel pack is removed and placed into a fresh pad. Obviously, this can be repeated indefinitely for any number of fresh absorbent pads.

The garments disclosed herein can be all natural, organic, or both. In certain exemplary embodiments the garment is impregnated or otherwise comprises an active agent. Non-limiting examples include a deodorant, perfume, desiccant powder or other drying agent, antiperspirant, antibacterial, anti-fungal, probiotic or a combination of any two or more of them. Anyone or more of the layers of the garments disclosed here may be formed of one or a combination of materials. Non-limiting examples of suitable materials will be readily apparent to those of ordinary skill in the garment art given the benefit of this disclosure and include numerous commercially available materials. Exemplary suitable materials include, woven, unwoven, knitted, lace, stitched, pressed, or other fabric or soft cloth, paper, silicone, hydrocolloid, or other suitable material(s) or the like, such as a single layer of natural fiber fabric, optionally organically grown natural fiber, e.g., cotton, flax, or the like, or a single layer of synthetic fiber fabric, e.g., rayon, polyester, nylon, spandex, sponge, foam (e.g. medical foam), Lycra or the like, or a single layer of mixed natural and synthetic fiber fabric. Other non-limiting examples of materials include resin, twill, canvas, burlap (hessian), silk, felt, wool, polypropylene, denim, leaves, fibers, weave, hemp, and/or a combination of one or more materials. For example, all or a portion of the garment may be formed of high twist cotton elastic cloth, e.g., with rubber-based, acrylic or other adhesive, optionally with a feathered edge. All or any portion(s) of any such materials of the web may be perforate or imperforate, porous or nonporous, moisture permeable, semi-permeable or impermeable, opaque, translucent and/or transparent. Correspondingly, all or anyone or more portions of the garment may be perforate or imperforate, porous or nonporous, moisture permeable or impermeable, opaque, translucent and/or transparent. The garment can preferably be hypoallergenic, i.e., for a majority of wearers cause no significant adverse dermatological reaction, e.g., no reaction beyond possibly a minor, temporary skin irritation in the area. In certain exemplary embodiments, the garment is permeable to oxygen, moisture and liquid. In certain exemplary embodiments, it is non-permeable. In certain exemplary embodiments it is semi-permeable, that is, the web is permeable to moisture but not to liquid. Certain exemplary embodiments are formed at least in part of such semi-permeable materials as Gore-Tex®. Certain exemplary embodiments comprise hydrocolloids. Certain embodiments of the garments disclosed here are bio-occlusive, either fully bio-occlusive or semi-bio-occlusive, that is, they prevent or substantially prevent or reduce odor from the vulva or crotch area.

Materials suitable for at least certain exemplary embodiments of the garments disclosed here that comprise a non-permeable layer include, for example, thin layers of polyvinyl chloride film or other vinyl material, polyethylene film, polyurethane film, silicone film, or any other suitable substance or material. Additional suitable materials will be readily apparent to those of ordinary skill in the art given the benefit of this disclosure. Certain exemplary embodiments of the garments disclosed here optionally comprise one or more active agents, e.g., a perfume, cologne, essential oils, favoring agent, pharmaceutical, nutraceutical, herbiceutical, nutrient, phytonutrient, probiotic, anti-viral, viricide, antibacterial, antiperspirant, anti-fungal, antiseptic, deodorant, odor neutralizer or suppressant, anti-microbial, microbicide, anti-itching agent, desiccant powder or other drying agent, cooling agent, vitamins, antioxidant, skin rejuvenating agent, pH stabilizing agent, libido enhancement and/or stimulant, hormonal balancing, sun protectant, lubrication and any other suitable agent(s) medically or cosmetically useful, or a mixture of any two or more of them. Exemplary antioxidants include gallic acid, Vitamin C, ascorbic acid, Vitamin E. Those of ordinary skill in the art will understand that there is overlap amongst the categories of active agents listed above. The optional active agent(s) may be provided in or on the web and/or the adhesive and/or any other component of the garment, including, optionally, for example, a replaceable component, as discussed above. In certain exemplary embodiments, the anti-itching agent comprises starch, e.g., cornstarch, kaolin, zinc oxide, such as Mexsana (an antiseptic medicated powder), Calamine (a mixture of zinc oxide and iron oxide), etc. In certain exemplary embodiments the active agent comprises an odor neutralizing agent selected from zinc and zinc compounds, silver and silver compounds, activated carbon, baking soda, etc., or a combination of any of them. Certain exemplary embodiments of the garments disclosed here are odor neutralizing and/or odor suppressing agents. Such embodiments optionally are also odor impermeable as described above. Odor neutralizing and/or suppressing embodiments may contain one or a combination of odor neutralizing agents, e.g., deodorant, antiperspirant, antibacterial agent, antifungal agent, odor absorbent, and/or other odor neutralizer. For example, suitable odor neutralizing and/or suppressing agent may be provided in the garment. Numerous odor-neutralizing agents and/or suppressing agents suitable for use in the garments disclosed here will be apparent to those skilled in the art given the advantage of this disclosure. Exemplary neutralizing agents and/or suppressing agents suitable for at least certain embodiments of the garments include activated charcoal, zinc oxide, sodium bicarbonate, fragrance, aluminum zirconium tetrachlorohydrate and/or other aluminum chlorohydrate, 5-chloro-2-(2,4-dichlorophenoxy) phenol) or other wide spectrum antibacterial and antifungal agent, etc. In certain exemplary embodiments the neutralizing agent and/or suppressing agent is time-released, that is, it is slowly released overtime to the wearer in a manner the same as or similar to drug delivery via well-known transdermal patch techniques, as the garment is being worn. At least certain such embodiments can advantageously provide continual freshness for the wearer. Rubber or other elastomeric materials can further be incorporated into the garments disclosed. One suitable material is a LYCRA® elastic material. For example, the various elastic elements can be formed of LYCRA® XA Spandex 540, 740 or 940 decitex T-127 or T-128 elastics available from DuPont, styrene copolymers of polyisoprene, polybutadiene or polyolefin, copolymers of polyolefins, natural or styrene butadiene rubber, polyurethanes, polyamides, polyesters, and co-extrusions/blends of the aforementioned materials. Elastomeric composite materials can included in the garment. Non-limiting examples, include a continuous filament stretch bonded laminate (CFSBL), a vertical filament laminate (VFL), neck-bonded-laminate (NBL), a stretch-bonded laminate (SBL), a necked-stretch bonded laminate (NSBL) or a necked-thermal laminate, or the like, as well as combinations thereof.

According to a specific embodiment, the instant garments, in addition to providing cover, warmth or cold, keep its wearers healthy and may be fitted with sensors powered by a tiny embedded lithium battery, and are washable. Garment-integrated sensors provide smart sensing capability, while preserving the comfort of the user. Sensors integrated directly into garments or fabric structures can also be used to detect the movement of the garment during wearing or to sense folds in the garment. In this configuration, the wearer's data can also be wirelessly transmitted to a central computer for analysis and feedback. The in-built sensors gather information on the wearer and responds accordingly. For example, heat or cold sensors measure temperature and then adjust accordingly in a feedback-type response. Other objectives of this invention is to provide smart garments for firefighters and rescuers that will help them and their headquarters to know the location, the capacity, and the encountered risks of emergency personnel on the intervention's field. In one specific embodiment of the instant invention, the smartness is given by the network of integrated sensors and the communication capacities of the developed garments wherein integrated life sign monitoring sensors for heart rate, respiration rate, body temperature, environmental risks (chemical, temperature), the situation of the rescuer (activity, attitude, localization) can be integrated.

It is understood that the invention is not limited to the disclosed methods, compositions and embodiments above, including any embodiments that may be apparent to one of ordinary skill in the art. It should also be understood that the various features described above could be used together in any combination or permutation. All such combinations and permutations, whether or not specifically, individually mentioned expressly in this disclosure or shown in the drawings are within the scope of the inventive subject matter.

Although the foregoing invention has been described in some detail, it will be readily apparent to those of ordinary skill in the art in light of the teachings of this invention that certain variations and modifications may be made thereto without departing from the spirit or scope of the disclosure herein, including the specific embodiments. Other embodiments are set forth within the following claims. Thus, while preferred embodiments of the invention have been illustrated and described, it is clear that the invention is not so limited. Numerous modifications, changes, variations, substitutions, and equivalents will occur to those skilled in the art without departing from the spirit and scope of the present invention as defined by the following claims. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. All changes, which come within the meaning and range of equivalency of the claims, are to be embraced within their scope.

What is claimed is:

1. A hypoallergenic garment comprising one or more interior compartments, said garment suitable for providing relief by achieving a desirable cooling or warming effect for an individual experiencing pain or discomfort, wherein said garment is impregnated with an active agent and comprises the following structural arrangement:
(i) one or more interior compartments within the garment where one or more warming or cooling pads containing a gel may be inserted and/or secured prior to use,
(ii) a pre-applied adhesive material on the body side of garment, suitable for contact with skin and sufficiently adherent to removably hold the pad(s) in place during use for at least more than one hour in the desired position while eliminating the need for cumbersome attachment mechanisms,
(iii) a removable backing layer for protecting the adhesive over at least a portion of said adhesive,
(iv) a thermal adjustment layer placed in varying locations relative to the pad(s) so as to help with temperature regulation,
(v) an absorbent layer designed to be placed in the garment against the skin that promotes fluid to wick across at least portions of its surface and assist in temperature regulation,
(vi) one or more sensors integrated into the fabric to provide smart sensing capability while preserving the comfort of the user,
wherein (i) said gel is thin, smooth, flexible and/or moldable to said individual's shape in order to maximize comfort and minimize/eliminate lumps or protruding regions, (ii) said pads may be placed in a cooling or warming environment prior to use or activated by folding or bending; and (iii) a discreet storage container housing said garment between uses.

2. The garment of claim 1, wherein said garment is for use in the groin and/or adjacent regions, is in pre-curved U-shaped or V-shaped configuration and contains additionally a flexible hydrophilic fabric or sponge reservoir present in the arrangement and functions as a means for fluid to migrate from the fabric thereto.

3. The garment of claim 1, wherein the gel comprises water, salt, hydroxy-ethyl cellulose, colorants, preservatives and anti-freeze.

4. The garment of claim 1, wherein the sensor is powered by a tiny embedded lithium battery, the wearer's data wirelessly transmitted to a central computer for analysis and feedback, wherein said sensor monitors heart rate, perspiration, respiration rate, body temperature, environmental risks, activity, attitude, localization, and combinations thereof.

5. The garment of claim 1, wherein said container containing the gel pads can be placed directly into a refrigerator, freezer or microwave and one or more pads used as needed.

6. The garment of claim 1, wherein said thermal adjustment layer, may be placed in varying locations relative to the pad so as to help with temperature regulation.

7. The garment of claim 1, wherein said pad has a length of about 6-9 inches and a width of about 2-3 inches.

8. The garment of claim 1, being permeable to oxygen, moisture and liquid.

9. The garment of claim 1, being permeable to moisture but not to liquid.

10. The garment of claim 1, wherein said active agent is selected from the group consisting of perfume, cologne, essential oil, favoring agent, pharmaceutical, nutraceutical, herbiceutical, nutrient, phytonutrient, probiotic, antiviral, viricide, antibacterial, antiperspirant, antifungal, antiseptic, deodorant, odor suppressant, antimicrobial, microbicide, anti-itching agent, desiccant, cooling agent, vitamin, antioxidant, skin rejuvenating agent, pH stabilizing agent, hormone, nanotherapeutic, therapeutic, sun protectant and combinations thereof.

11. The garment of claim 1, wherein said cooling pad is elongated to provide and wherein the corners of the pad are rounded in order to prevent them from poking or otherwise irritating the user's body during placement and use.

12. The garment of claim 1, wherein said garment is for treatment of conditions selected from the group consisting of itching, burning, yeast or bacterial infection, urethral pain, inflammation, urinary tract infections, dermatitis, vaginal varicosity, menopausal perspiration, post-surgery swelling, edema due to a sports injury, swelling due to trauma, damage to the perineum, menopausal pain and combinations thereof.

13. The garment of claim 1, wherein the gel is formulated to remain flexible or moldable at a temperature in a range of about 0° F. to about 110° F.

14. The garment of claim 1, wherein the gel can be repeatedly placed in a cooling or warming environment prior to use and will subsequently provide a cooling or warming effect for a period of time of at least about 15 minutes when the pad is placed in the garment and used against a user's skin.

15. The garment of claim 1, further contains (i) a plurality of thermally conductive elements, each promoting fluid to absorb into and wick across at least portions of a surface or surfaces of the pad by capillary action, (ii) flexible or elastic means interconnecting said thermally conductive elements, (iii) means for wetting the fluid-wicking surfaces of said thermally conductive elements in contact with the user's skin, whereby when said thermally conductive elements are wet and one or more portions of them are in contact with the user's skin, heat from the user's skin is transferred through said thermally conductive elements, to the evaporating fluid, and into the surrounding air, thereby cooling the user.

16. The garment of claim 15 further contains an air space parallel and adjacent to the user's skin and a fan for moving air through the air space may be incorporated to speed the evaporation of fluid from the surfaces of the elements.

17. A method of treating pain or discomfort of the groin and/or adjacent regions, wherein said pain or discomfort is a result of a condition selected from the group consisting of itching, burning, yeast or bacterial infection, urethral pain, inflammation, urinary tract infections, dermatitis, vaginal varicosity, menopausal perspiration, post-surgery swelling, edema due to a sports injury, swelling due to trauma, damage to the perineum, menopausal pain and combinations thereof,
wherein said method comprises using a hypoallergenic garment having the following structural arrangement:
(i) one or more interior compartments within the garment where one or more warming or cooling pads containing a gel may be inserted and/or secured prior to use,
(ii) a pre-applied adhesive material on the body side of garment, suitable for contact with skin and sufficiently adherent to removably hold the pad(s) in place during use for at least more than one hour in the desired position while eliminating the need for cumbersome attachment mechanisms,
(iii) a removable backing layer for protecting the adhesive over at least a portion of said adhesive,
(iv) a thermal adjustment layer placed in varying locations relative to the pad(s) so as to help with temperature regulation,
(v) an absorbent layer designed to be placed in the garment against the skin that promotes fluid to wick across at least portions of its surface and assist in temperature regulation,
(vi) one or more sensors integrated into the fabric to provide smart sensing capability while preserving the comfort of the user, and wherein said method involves the following steps performed sequentially:
(i) placing one or more pads into a cooling environment or microwave; (ii) removing a protective cover from a pre-applied adhesive material that is present on said garment,
(iii) inserting one or more said pads into one or more compartments within said garment,
(iii) wearing the garment for at least one hour in the desired position, and optionally
(iv) repeating steps (i) to (iii) as needed to obtain relief.

18. The method of claim 17, wherein the sensor is powered by a tiny embedded lithium battery, the wearer's data wirelessly transmitted to a central computer for analysis and feedback, wherein said sensor monitors heart rate, perspiration, respiration rate, body temperature, environmental risks, activity, attitude, localization, and combinations thereof

19. The method of claim 18, wherein the sensor further has heating or cooling elements incorporated therein and has a mechanism whereby it can heat or cool the gel in use, based on the wearer's data received from a central computer, thereby maintaining the garment at a predetermined temperature range.

20. A hypoallergenic undergarment suitable for providing relief by achieving a desirable cooling effect for women experiencing pain or discomfort of the vulva and/or adjacent regions, wherein said undergarment consists essentially of the following structural arrangement:
(i) an undergarment impregnated with an active agent selected from the group consisting of perfume, cologne, essential oil, favoring agent, pharmaceutical, nutraceutical, herbiceutical, nutrient, phytonutrient, probiotic, antiviral, viricide, antibacterial, antiperspirant, antifungal, antiseptic, deodorant, odor suppressant, antimicrobial, microbicide, anti-itching agent, desiccant, cooling agent, vitamin, antioxidant, skin rejuvenating agent, pH stabilizing agent, hormone, nanotherapeutic, therapeutic, sun protectant, and combinations thereof,
(ii) a pre-applied adhesive material on the body side of undergarment, suitable for contact with skin and sufficiently adherent to removably hold the cooling pad in place during use for more than one hour in the desired position while eliminating the need for cumbersome attachment mechanisms,
(iii) a removable protective cover over at least a portion of said adhesive,
(iv) a thermal adjustment layer placed in varying locations relative to the cooling pad so as to help with temperature regulation,
(v) an absorbent layer designed to be placed against the vulva and/or adjacent regions that promotes fluid to wick across at least portions of its surface,
(vi) one or more sensors integrated into the undergarment to provide smart sensing capability while preserving the comfort of the user, the sensor powered by a tiny embedded lithium battery, the wearer's data wirelessly transmitted to a central computer for analysis and feedback, said sensor monitors heart rate, perspiration, respiration rate, body temperature, environmental risks, activity, and combinations thereof,
wherein (i) said cooling gel is thin, smooth, flexible and/or moldable to the female shape in order to maximize comfort and minimize/eliminate lumps or protruding regions, (ii) said cooling pads can be placed in a cooling environment prior to use or cooling can be activated by folding or bending said pads; and (iii) a discreet storage container houses said undergarment,
and wherein said undergarment is for treatment of conditions selected from the group consisting of itching, burning, yeast or bacterial infection, urethral pain, inflammation, urinary tract infections, dermatitis, vaginal varicosity, menopausal perspiration, post-surgery swelling, edema due to a sports injury, swelling due to trauma, damage to the perineum, menopausal pain and combinations thereof.
